(19) United States
(12) Patent Application Publication
(10) Pub. No.: US 2008/0154181 A1
(43) Pub. Date: Jun. 26, 2008

(54) Central nervous system ultrasonic drain

(76) Inventor: Rohit K. Khanna, (US)

Correspondence Address: ROHIT KHANNA;SUITE 580, 311 N. CLYDE MORRIS BLVD., DAYTONA BEACH, FL, 32114, US

(21) Appl. No.: 12/008,611

(22) Filed: Jan. 11, 2008

Related U.S. Application Data
(63) Continuation-in-part of application No. 11/418,849, filed on May 5, 2006.

(51) Int. Cl. A61B 17/22

(52) U.S. Cl. 604/22

(57) ABSTRACT

The invention provides a method and apparatus for treating hemorrhage and maintaining catheter patency in the brain and spine through a new and minimally invasive technique. Ultrasound energy is delivered either through a catheter inserted directly into the hemorrhage and the delivered ultrasound energy dissolves the blood clot which is then drained through the catheter.

### BACKGROUND OF THE INVENTION

Intracranial hemorrhage has a very prevalent incidence and occurs in 13% of strokes and 23% of head injuries. It accounts for almost 20% of all deaths due to strokes and 72% of deaths from trauma. The impact on health care as well as loss of productivity and consequent disability cost the society several billions of dollars each year. The treatment of head injuries has lacked any remarkable progress in the past several decades. Fortunately, several advances have been made in the treatment for strokes. A stroke is caused by occlusion of a blood vessel supplying blood flow to the brain usually by a blood clot inside the vessel. Treatment strategies have focused on dissolving this blood clot inside the blood vessel. These include the use of thrombolytic agents like tissue plasminogen activators (t-PA) and intravascular catheters that use mechanical disruption, ultrasonic or photonic heat energy to dissolve blood clots occluding the cerebral blood vessels. Although these treatment advances address intravascular blood clot hemolysis for ischemic stroke, none of these offer treatment for subdural or cerebral hemorrhage (hemorrhagic stroke), which is the predominant cause of morbidity and mortality in these patients.

Surgery has generally been advocated for evacuation of intracranial hemorrhages which are large enough to cause brain swelling or neurologic deficits. In most medical centers, the usual delay between the time the hemorrhage is detected until the surgical intervention is undertaken can be several hours. This delay is not always preventable since surgery requires preparation of the operating room with its equipment and personnel, anesthesia induction, and creating a large opening in the skull via a craniotomy to expose the brain and evacuate the hemorrhage. For hemorrhages located in the deeper structures of the brain, surgery requires extensive manipulation through the normal part of the brain to expose and evacuate the hemorrhage. For treatment of intra-ventricular hemorrhage, current methodology teaches placement of a ventriculostomy drain through a burr hole created in the skull. Unfortunately, acute hemorrhage turns into a blood clot within a few minutes and therefore, does not drain out through a tube until it dissolves. This natural blood clot dissolution process can take several days to weeks.

Also, a ventriculostomy drain almost always gets obstructed from the blood clots which, in turn also foster infectious complications. Consequently, there remains a great margin for improvement, particularly with treatment options providing for a faster, less invasive, and a low complication approach for central nervous system hemorrhage. Several ultrasonic devices have been proposed in the prior art and all of these have focused on dissolution of intravascular blood clots. These include catheters placed intravascularly to help dissolve the blood clot occluding the vessel or externally placed therapeutic ultrasound probes. While ischemic stroke results from occlusion of a cerebral blood vessel from a blood clot, intracranial hemorrhage consists of a blood clot that is outside the intracranial blood vessels and inside the brain or skull. There is no description in the prior art for treatment of intracranial or spinal subdural or subarachnoid hemorrhage with the use of ultrasonic devices.

The use of ultrasound therapy to dissolve blood clots is well described in U.S. Pat. No. 4,441,486, Hall et al., U.S. Pat. No. 5,460,595, Unger et al., U.S. Pat. No. 5,558,092, and Chapelon et al., U.S. Pat. No. 5,601,526. DonMicheal et al., U.S. Pat. No. 4,870,953, Guess et al., U.S. Pat. No. 5,069,664, Carter, U.S. Pat. No. 5,269,291, Marcus et al., U.S. Pat. No. 5,295,484, Hashimoto, U.S. Pat. No. 5,307,816, Carter, U.S. Pat. No. 5,362,309, Carter U.S. Pat. No. 5,431,663, and Rosenschein, U.S. Pat. No. 5,524,620. U.S. Pat. No. 6,635,017 to Moehring, et al.

The interaction between ultrasound and a thrombolytic agent has been shown to assist in the break-down or dissolution of a blood clot, as compared with the use of the thrombolytic agent alone. This phenomenon is discussed, e.g., in Carter U.S. Pat. No. 5,509,896; Siegel et al U.S. Pat. No. 5,695,460; and Lauer et al U.S. Pat. No. 5,399,158, which are each incorporated herein by reference.

### SUMMARY OF THE INVENTION

For the treatment of intracranial hemorrhage, an ideal methodology would allow for evacuation of the hemorrhage through a minimally invasive approach which can be undertaken at the bedside either in the emergency room or intensive care unit and without the need for general anesthesia. Minimizing the surgical intervention delay as well as well as avoiding going through normal parts of the brain to get to the hemorrhage provides for better outcomes and reduced mortality.

The present invention describes methodology for the treatment of intracranial hemorrhage. Ultrasonic energy is used to hemolyse and dissolve the blood clot. This can be achieved through placement of an ultrasound delivery catheter directly into the hemorrhage. The clot hemolysis can be facilitated with the use of thrombolytic, hemolytic, antiplatelet, and/or anticoagulant agents also delivered through the catheter. The dissolved clot is then drained through the catheter either via dependent gravity drainage or a suction apparatus. Placement of the catheter utilizes a well versed “burr hole” technique commonly practiced in the field of neurosurgery for placement of ventriculostomy catheters and cerebral pressure monitoring devices. Typically, a small skin incision is made in the head using standard external landmarks. A small hole in the skull is then created with the use of a drill and subsequently a catheter is then placed into the brain or subdural space. A precise placement of the catheter can be facilitated with the use of stereotactic techniques if needed.

Ultrasonic energy focused upon a blood clot causes it to break apart and dissolve. This process termed thrombolysis liquefies the clot and allows subsequent drainage via a catheter or even absorption by the brain. Depending on the frequency of the ultrasonic energy used, the ultrasound effect is carried through by means of mechanical action, heat, or cavitation. The lower frequency acoustical waves, usually below 50 KHz, dissolve a blood clot by cavitation and frequencies above 500 KHz take affect more so by generating heat. These waves can be focused to produce a therapeutic effect up to 10 cm or more from the transducer.

The ultrasonic frequency waves can also be generated continuously or in a pulsed format. Use of continuous waves allows clot dissolution in a shorter time period but also generates more heat. Pulsed waves prevent heat build-up and reduce the risk of cavitation in the target tissue, but may also take affect over a longer period of time. For example, at frequencies in the range from 50 to 150 MHz, dissolution only occurs in close proximity to the face of the transducer with the actual distance depending upon the elastic and acoustical properties of the propagating medium. Adverse rises in temperature are also prevented, preferably by selecting a pulsed mode of operation, such that coagulation of tissue and other disadvantageous side-effects accompanying adverse temperature rises can be avoided. Applying ultra-high frequency energy 50 MHz to 100 GHz) to the hemorrhage in pulses, rather than as a continuous wave, may actually reduce the time required to dissolve tissue structures; however continuous wave application is also effective. In pulsed mode operation, for example in pulses of about 10 to about 100 wavelengths in duration, substantially higher wave amplitudes, but lower energy densities, can be applied to the hemorrhage with the assurance that any high-frequency vibratory mode imparted to the hemorrhage by the acoustical waves will also be absorbed within the localized area of the target tissue.

Whereas relatively low frequency ultrasonic devices break apart the hemorrhage by mechanical impact or cutting action, a radiated propagating wave of high frequency ultrasonic energy, preferably in short pulses, dissolves blood clots into its cellular/sub cellular components in a highly controlled and localized manner.

In some instances, cooling may be needed to avoid the adverse effects of temperature rises by ultra-high frequency energy use. Several methodologies and cooling catheters have been described in U.S. patent application Ser. No. 11/418,849.

Ultrasound frequency in the 100 MHz range can be used to dissolve blood clots in a very localized region within 1 mm of the transducer without deleteriously affecting the surrounding brain. By contrast, acoustical waves at 1 MHz travel about 3 cm before attenuation reduces its power by one half.

Similarly, wavelength helps to determine the type of destructive forces that operate in target material and the size of the particles generated. When the wavelength of sound is relatively long, cavitation and/or gross mechanical motion produce the blood clot break-up. Such a situation certainly exists if the frequency of the sound is around 40 kHz or below. When, however, the wavelength of sound is very much smaller, as it is at 100 MHz, the mechanical energy associated with the propagating sound wave breaks down the blood clot into cellular or sub cellular components. The depth of material breakdown as measured from the surface of the material to be treated is frequency dependent and the blood clot can be dissolved to a microscopic level by selecting the appropriate frequency. It has also been shown that a 100 MHz ultrasound frequency can dissolve blood clots by using a pulsed sequence without cavitation or heat generation using mainly a mechanical breakdown effect.

The process by which thrombolysis is affected by use of ultrasound in conjunction with a thrombolytic agent can vary according to the frequency, power, and type of ultrasonic energy applied, as well as the type and dosage of the thrombolytic agent. The application of ultrasound has been shown to cause reversible changes to the fibrin structure within the thrombus, increased fluid dispersion into the thrombus, and facilitated enzyme kinetics. These mechanical effects beneficially enhance the rate of dissolution of thrombi. In addition, ultrasound induced cavitational disruption and heating/streaming effects can also assist in the breakdown and dissolution of thrombi.

The thrombolytic agent can comprise a drug known to have a thrombolytic effect, such as streptokinase, urokinase, prourokinase, ancrod, tissue plasminogen activators (alteplase, anistreplase, tenecteplase, reteplase, duteplase. Alternatively (or in combination), the thrombolytic agent can comprise an anticoagulant, such as heparin or warfarin; or an antiplatelet drug, such as a GP IIb IIIa, aspirin, ticlopidine, clopidogrel, dipyridamole; or a fibrinolytic drug such as aspirin. Alternatively the thrombolytic agent can be incorporated into micro bubbles, which can be ultrasonically activated after direct infusion into the blood clot through a catheter.

It may be possible to reduce the typical dose of thrombolytic agent when ultrasonic energy is also applied. It also may be possible to use a less expensive or a less potent thrombolytic agent when ultrasonic energy is applied. The ability to reduce the dosage of thrombolytic agent, or to otherwise reduce the expense of thrombolytic agent, or to reduce the potency of thrombolytic agent, when ultrasound is also applied, can lead to additional benefits, such as decreased complication rate, and an increased patient population eligible for the treatment.

Catheters capable of delivering ultrasonic energy can be placed directly into the hemorrhage inside the skull, brain, or spine and facilitate blood clot dissolution and drainage. In some embodiments of the drainage catheters, ultrasonic energy generated outside the catheter is transmitted through conductors in the catheter wall or lumen. In other embodiments of the drainage catheters, ultrasonic energy is generated by transducers within the catheter.

Placement of a subdural drain following either a burr hole placement or craniotomy for evacuation of intracranial hemorrhage is a very common methodology practiced in neurosurgery. This drain is very prone to obstruction from the hemorrhage and not infrequently requiring further surgery to evacuate the residual or recurrent hemorrhage development. As described in the current methodology, a drain equipped with delivering ultrasonic energy to the lumen will also dissolve any obstruction from blood clots or debris in the lumen and significantly reduce this complication by maintaining drain patency.

### BRIEF DESCRIPTION OF THE DRAWINGS

FIG. 1 is a schematic view of the ultrasonic catheter in the brain.

FIG. 2 is a cross-sectional longitudinal view of one embodiment of the catheter.

FIG. 3 is a cross-sectional longitudinal view of another embodiment of the catheter.

FIG. 4 is a cross-sectional transverse view of the catheter taken along line A in FIG. 2.

FIG. 5 is a cross-sectional view of the catheter taken along line B in FIG. 3.

FIG. 6 is a cross-sectional side view of another embodiment of the catheter.

FIG. 7 is another cross-sectional side view of another embodiment of the catheter in.

FIG. 8 is a cross-sectional view of the catheter taken along line A in FIG. 6.

FIG. 9 is a cross-sectional view of the catheter taken along line A in FIG. 6.

FIG. 10 is a cross-sectional side view of another embodiment of the catheter.

FIG. 11 is a cross-sectional side view of another embodiment of the catheter.

FIG. 12 is a cross-sectional view of the catheter taken along line A in FIG. 11.

FIG. 13 is a cross-sectional view of the catheter taken along line B in FIG. 11.

FIG. 14 is a cross-sectional side view of another embodiment of the catheter.

FIG. 15 is a cross-sectional side view of another embodiment of the catheter.

FIG. 16 is a cross-sectional view of the catheter taken along line B in FIG. 14.

FIG. 17 is a cross-sectional view of the catheter taken along line A in FIG. 14.

FIG. 18 is a cross-sectional side view of another embodiment of the catheter.

FIG. 19 is a cross-sectional side view of another embodiment of the catheter.

FIG. 20 is a cross-sectional view of the catheter taken along line A in FIG. 18.

FIG. 21 is a cross-sectional view of the catheter taken along line A in FIG. 19.

FIG. 22 is a cross-sectional view of the catheter taken along line B in FIG. 19.

FIG. 23 is a cross-sectional side view of another embodiment of the catheter.

FIG. 24 is a cross-sectional side view of another embodiment of the catheter.

FIG. 25 is a cross-sectional side view of another embodiment of the catheter.

FIG. 26 is a cross-sectional view of the catheter taken along line A in FIG. 24.

FIG. 27 is a cross-sectional side view of another embodiment of the catheter.

FIG. 28 is a cross-sectional side view of another embodiment of the catheter.

FIG. 29 is a cross-sectional view of the catheter taken along line A in FIGS. 27 & 28.

### DETAILED DESCRIPTION OF THE PREFERRED EMBODIMENTS

In one method of intracranial hemorrhage treatment, a catheter 5 as shown in FIG. 1 can be placed into the brain 2 or ventricle 3 or the subdural or epidural space depending on the location of the hemorrhage. This catheter can be placed using the standard landmarks or can be precisely placed with stereotactic guidance or use of an endoscope. A bolt 4 can also be used to secure the catheter through the skull 1 but is not necessary. The catheter is placed either through a small drill hole created in the skull or after a craniotomy or burr hole placement.

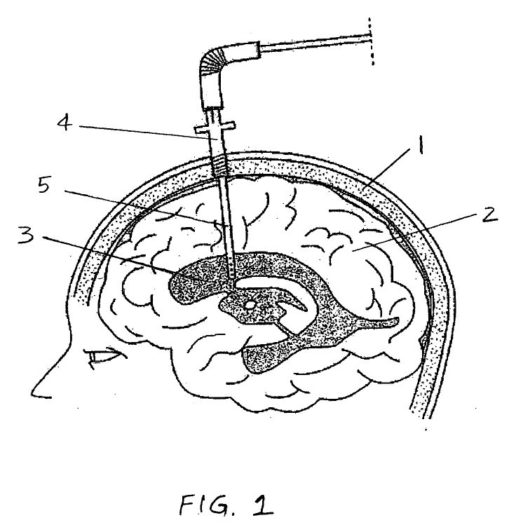

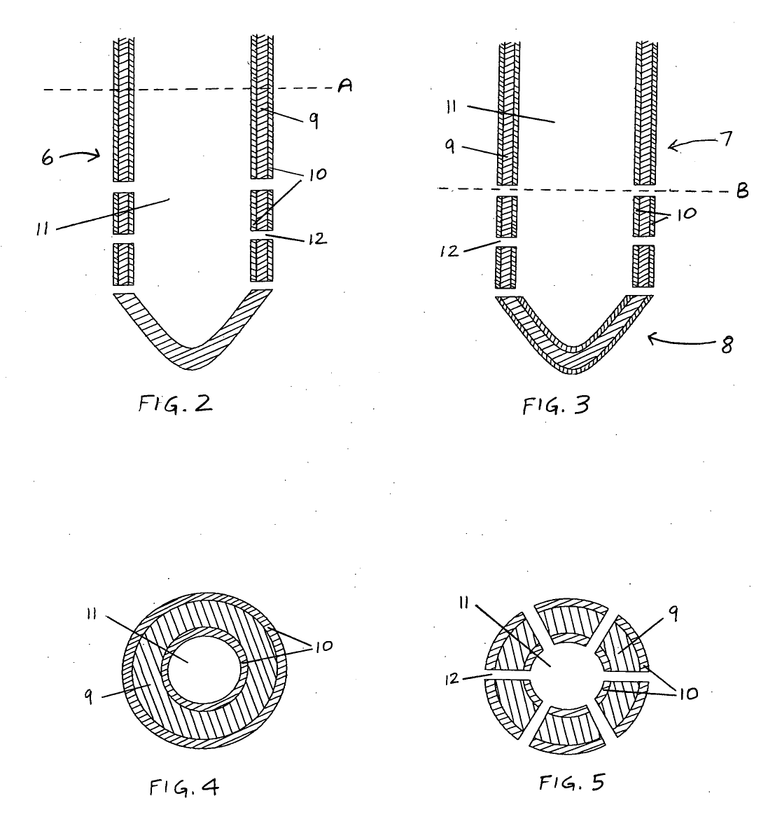

FIGS. 2-5 illustrate one embodiment of the ultrasonic catheter drainage system. The distal catheter wall 6 as seen in FIG. 2 or the wall 7 and tip 8 as seen in FIG. 3 contain the ultrasound transducer with a piezoelectric crystal 9 surrounded by electrodes 10. The catheter contains a lumen 11 with ports 12 at the distal ends that communicate with the external environment. When the catheter is placed directly into the blood clot, the ultrasonic energy dissolves the clot, which can be further facilitated if needed by infusing a hemolytic or thrombolytic or antiplatelet agent through the lumen and then draining the liquefied blood through the same lumen. Since the lumen communicates with the brain, it can also be used to monitor the intracranial pressure.

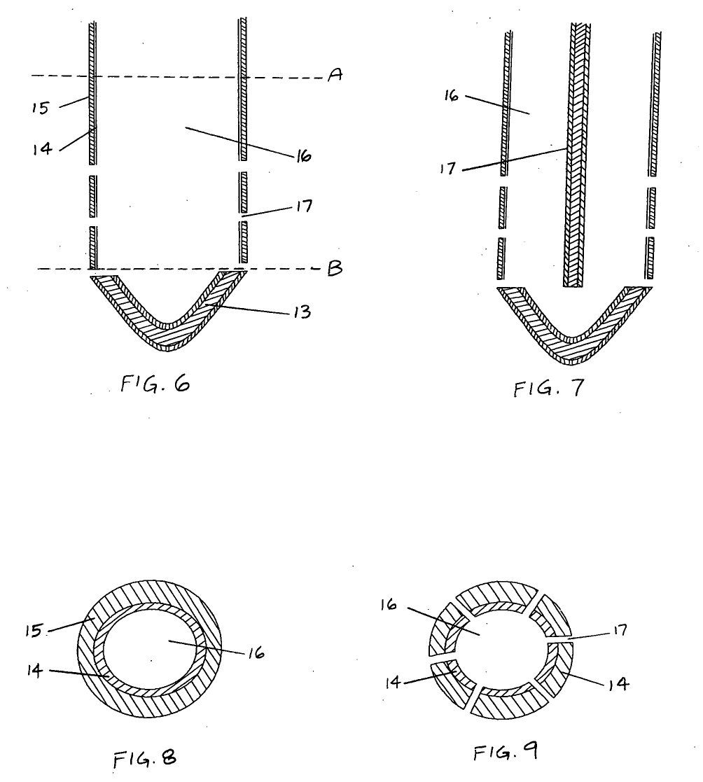

FIGS. 6-9 illustrate an ultrasonic catheter with the transducer 13 at the distal tip. The ultrasound transducer electrodes 14 are embedded in the catheter wall 15. The catheter contains a lumen 16 with ports 17 at the distal end that communicate with the outside environment. As shown in FIG. 7, the lumen 16 can also contain an ultrasound transducer 17 which is removable.

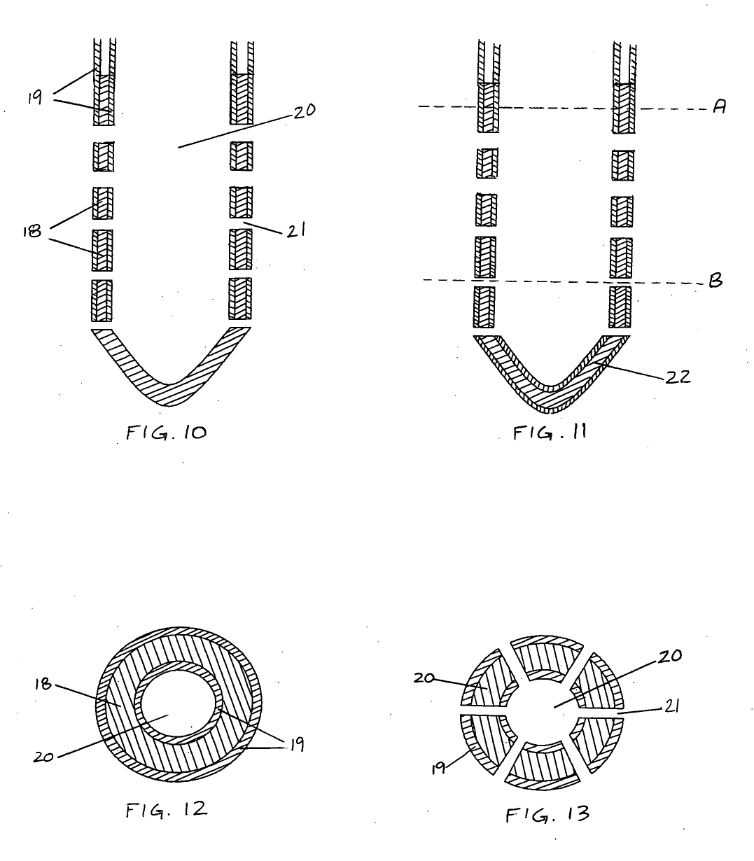

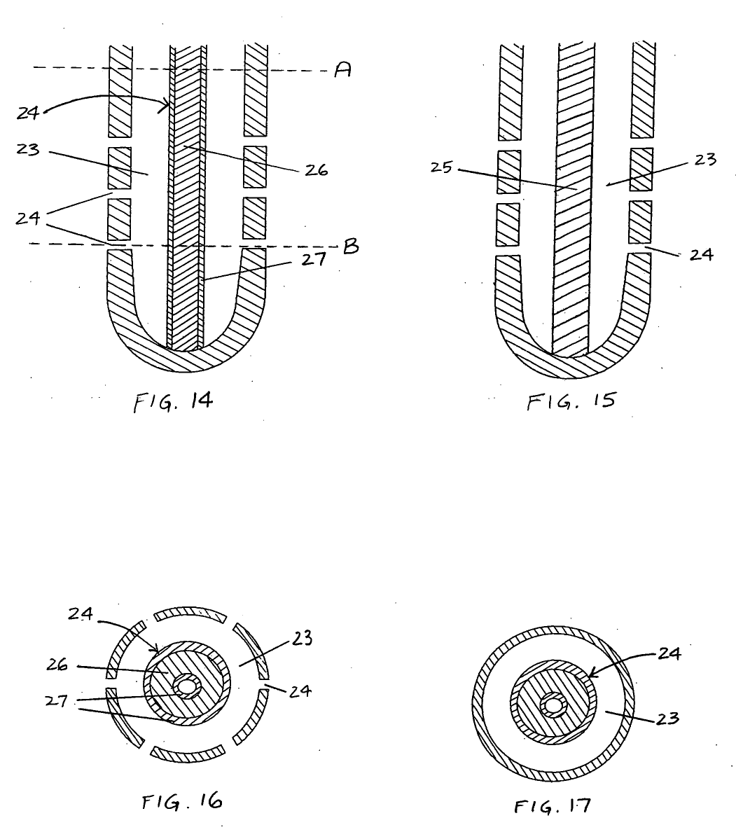

FIGS. 10-13 illustrate an ultrasonic catheter with the distal end comprising of a plurality of ultrasound transducers 18 connected to a signal generator at the proximal end through an electrical conductor 19. The catheter also has a longitudinal lumen 20 with portals 21 at the distal end. The ultrasound transducers also having a plurality of resonant frequencies and can receive a multi-frequency driving signal to the plurality of ultrasound transducers. I an another embodiment, the catheter tip 22 as shown in FIG. 11 also contains an ultrasound transducer.

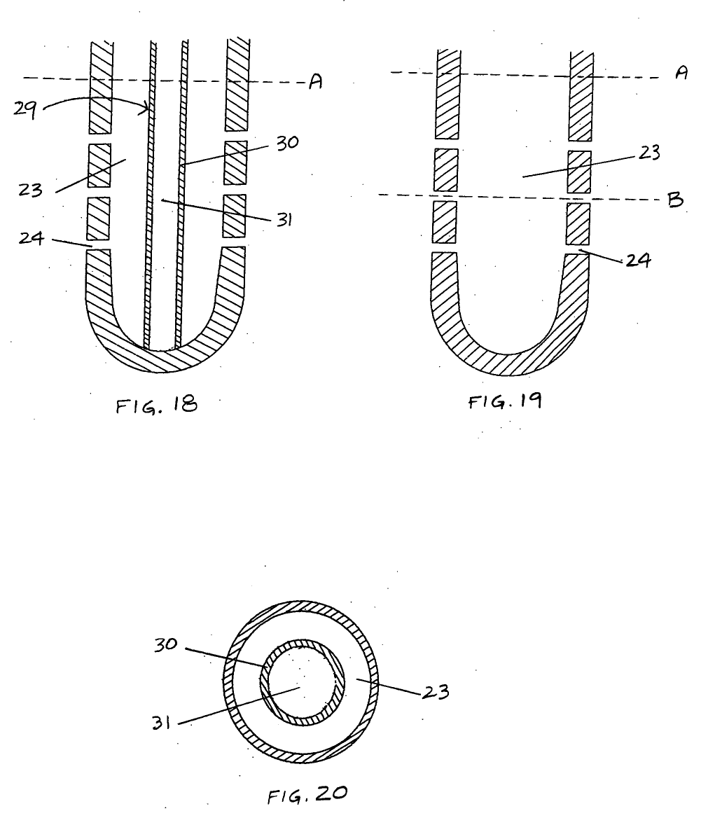

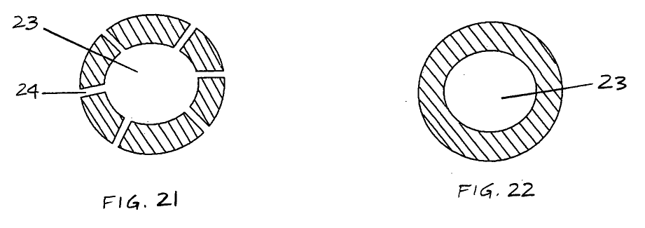

In another embodiment of the ultrasonic catheter as illustrated in FIGS. 14-22, the catheter contains a lumen 23 which communicates with the outside environment through ports 24. The lumen 23 is also capable of incorporating an ultrasound transducer 24 or conductor 25 which is removable. FIGS. 14, 16, & 17 illustrate a catheter with an ultrasound transducer 24 in the lumen 23. The transducer consists of a piezoelectric crystal 26 surrounded by electrodes 27. The ultrasound transducer 24 can be inserted or removed as needed for thrombolysis. FIG. 15 illustrates a catheter with an ultrasound conductor 25 in the lumen 23. The conductor 28 typically is comprised of a metal that transmits ultrasound energy from a generating source at the proximal end of the catheter. FIGS. 18 & 20 illustrate the catheter with an ultrasound conductor 29 in the lumen 23. The conductor 29 has a wall 30 and a lumen 31 filled with a fluid or gel that propagates ultrasonic waves through the catheter from a generating source connected to the proximal end of the catheter. FIGS. 19, 21, & 22 illustrate the catheter with the transducers removed from the lumen 23.

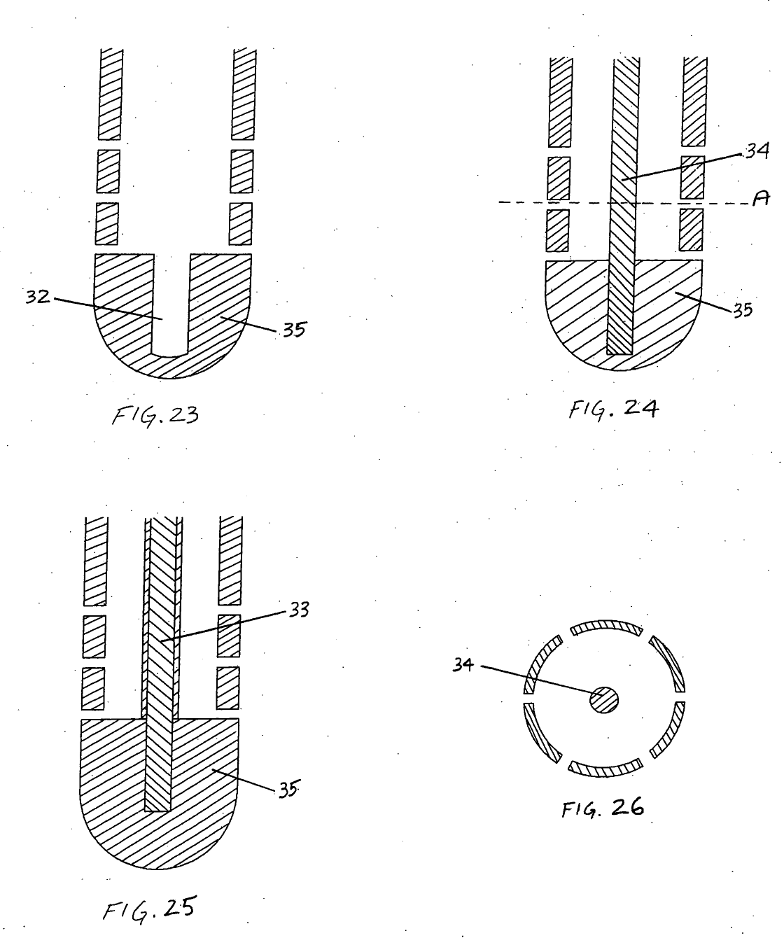

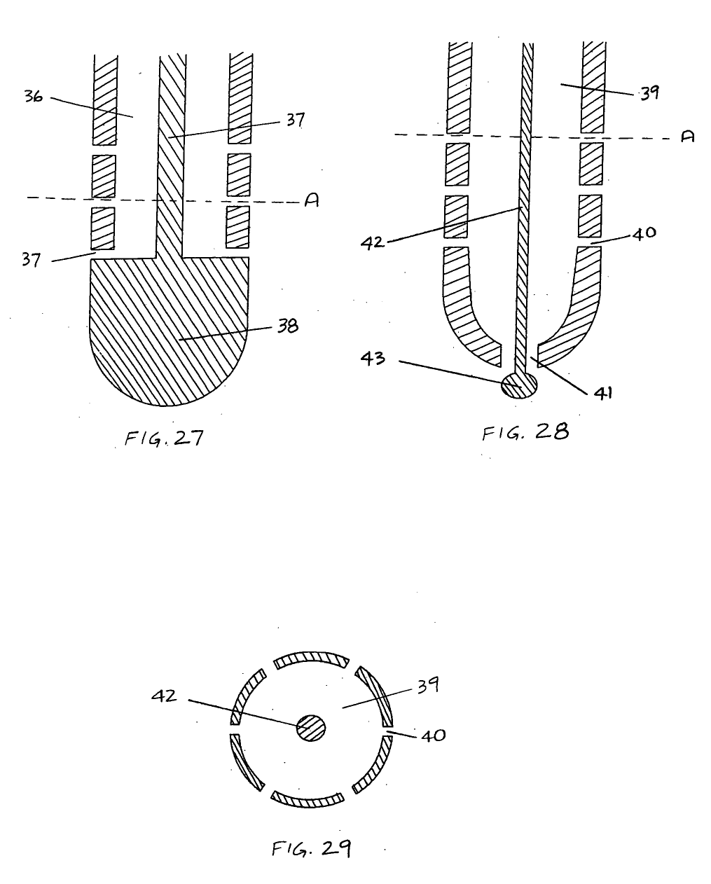

FIGS. 23-26 illustrate another embodiment of the catheter with an anchor 32 at the distal end for the removable ultrasound transducer 33 or conductor 34. This anchor can also serve as an amplifier 35 for the ultrasound energy. FIG. 23 illustrates the catheter with the ultrasound transducer removed.

FIG. 27 illustrates another embodiment of the catheter with a lumen 36 and ports 37 at the distal end. The lumen 36 contains an ultrasound conductor 37 attached to an amplifier 38 at the tip. Ultrasonic energy is generated from an outside source and transmitted through the conductor and is further amplified by the amplifier at the catheter distal end. FIGS. 28 & 29 illustrate another embodiment of the catheter with a lumen 39 and ports 40 at the distal end and an opening 41 at the tip. The lumen 39 contains an ultrasound conductor 42. The conductor 42 has an enlarged distal end 43 that can extend outside the catheter lumen 39 through the opening 41. The enlarged distal conductor end amplifies the ultrasound energy as well as facilitates blood clot hemolysis extending outside the catheter tip.

While the methodology described herein is specific for central nervous system hemorrhage treatment and prevention of catheter obstruction, its use is not limited to this particular pathology. These catheters can also be used to treat various other central nervous system pathologies. For instance, ultrasonic energy directly transmitted into a brain tumor with the catheter system allows tumefaction and dissolution of the tumor cells which can then be drained directly.

Similarly the tumefaction process can be facilitated with a direct delivery of a chemotherapeutic agent through the catheter.

1. A method of treating a hemorrhage in the central nervous system wherein ultrasound energy is used to dissolve the said hemorrhage comprising the steps of: inserting a catheter into the hemorrhage; delivering ultrasound energy through the said catheter; draining the dissolved hemorrhage through a lumen in the said catheter.

2. The method of claim 1 wherein the said catheter comprising of a proximal and distal end and two lumens which extend longitudinally from the proximal to the distal end; the proximal end of one lumen comprising of an ultrasound transducer to propagate ultrasound waves through the fluid or gel filled lumen to the distal end of the catheter; the distal end of the other lumen comprising of one or more portals for drainage.

3. The method of claim 1 wherein the said catheter comprising of a proximal and distal end and a lumen which extends longitudinally from the proximal to the distal end; the proximal end comprising of an ultrasound transducer which conducts ultrasonic energy through one or more metal wires embedded in the wall to the distal end; the distal end of the lumen comprising of one or more portals.

4. The method of claim 1 wherein the said catheter comprising of a proximal and distal end; the distal end comprising of an ultrasound transducer connected to a signal generator at the proximal end through an electrical conductor; the said catheter also comprising of a longitudinal lumen with one or more portals at the distal end.

5. The method of claim 1 wherein the said catheter comprising of a proximal and distal end; the distal end of the catheter wall comprising of multiple ultrasound transducers connected to a signal generator at the proximal end through an electrical conductor; the said catheter also comprising of a longitudinal lumen with one or more portals at the distal end.

6. The method of claim 1 wherein the said catheter comprising of a proximal and a distal end with a lumen; the distal end wall of the catheter comprising of an ultrasound transducer with one or more portals communicating from the outer environment to the lumen.

7. The method of claim 1 wherein the said catheter comprising of a proximal and a distal end with a lumen; the distal end wall of the catheter comprising of an ultrasound transducer with one or more portals communicating from the outer environment to the lumen; the lumen also comprising of an ultrasound transducer.

8. The method of claim 7 wherein the said ultrasound transducer in the lumen is removable.

9. The method of claim 1 wherein the said catheter comprising of a proximal and a distal end with a lumen with one or more portals at the distal end that communicate with the external environment; the lumen comprising of an ultrasound transducer.

10. The method of claim 9 wherein the said ultrasound transducer in the lumen is removable.

11. The method of claim 1 wherein the said catheter comprising of a proximal and a distal end with a lumen with one or more portals at the distal end that communicate with the external environment; the lumen comprising of an ultrasound transducer at the center with an amplifier at the distal end.

12. The method of claim 1 wherein the said catheter comprising of a proximal and distal end and a lumen which extends longitudinally from the proximal to the distal end; the proximal end comprising of an ultrasound transducer which conducts ultrasonic energy through one or more metal wires to an amplifier at the distal end; the distal end of the lumen comprising of one or more portals to drain the dissolved hemorrhage.

13. The method of claim 1 wherein the said catheter comprising of a proximal and distal end and a lumen which extends longitudinally from the proximal to the distal end; the proximal end comprising of an ultrasound transducer which conducts ultrasonic energy through a solid metal to an amplified distal end; the distal end of the lumen comprising of one or more portals to drain the dissolved hemorrhage and also allow extension of the ultrasound conductor outside the catheter.

14. The method of claim 1 wherein the said catheter comprising of a proximal and distal end and a lumen which extends longitudinally from the proximal to the distal end; the proximal end comprising of an ultrasound transducer which transmits ultrasonic energy through a conductor to the distal end; the distal end of the lumen comprising of one or more portals to drain the dissolved hemorrhage.

15. The method of claim 1 wherein the said central nervous system comprises of one or more of the following: ventricle, subdural, subarachnoid, epidural, intra-cerebral, intra-thecal, brain, spine, skull, spinal cord.

16. The method of claim 1 further comprising the steps of hemorrhage dissolution facilitated by one or more of the following agents infused through the said catheter: i) thrombolytics like streptokinase, urokinase, prourokinase, ancrod, tissue plasminogen activators (alteplase, anistreplase, tenecteplase, reteplase, duteplase), ii) hemolytic agents, iii) antiplatelet agents like GP IIb IIIa, aspirin, ticlopidine, clopidogrel, dipyridamole, iv) anticoagulants like heparin or warfarin, v) fibrinolytic agent like aspirin, vi) thrombolytic agent incorporated into micro-bubbles which can be ultrasonically activated after direct infusion into the blood clot.

17. The method of claim 1 wherein, the catheter wall is impregnated with antimicrobial and/or anti-clotting agents comprising of one or more of the following: antibiotics, antifungal, iodine, metals, polymeric material, nitric oxide, antibodies, anticoagulant, anti-platelet, thrombolytic, chlorhexidine gluconate, anti-inflammatory.

18. The method of claim 1 wherein the said catheter is inserted into the hemorrhage with use of one or more of the following: i) ultrasound imaging, ii) stereotactic guidance, iii) endoscopy.

19. The method of claim 1 further comprising the steps of draining hemorrhage through a lumen in the said catheter with either a suction system or vacuum bulb or drainage bag attached to the distal end of the catheter.

20. The method of claim 19 wherein the said catheter shape is oval or rectangular.

21. The method of claim 19 wherein the said catheter is used as a drain following cranial or spinal surgery for drainage of post-operative hemorrhage.

22. The method of claim 1 wherein the said catheter comprising of a proximal and distal end; the distal end comprising of a plurality of ultrasound transducers connected to a signal generator at the proximal end through an electrical conductor; the said catheter also comprising of a longitudinal lumen with one or more portals at the distal end to drain the dissolved hemorrhage; the ultrasound transducers also having a plurality of resonant frequencies; and delivering a multi-frequency driving signal to the plurality of ultrasound transducers.

23. The method of claim 22 wherein the frequencies comprising the multi-frequency driving signal can vary with time, such that a first subset of ultrasound transducers emit ultrasonic energy during a first treatment period, and a second subset of ultrasound transducers emit ultrasonic energy during a second treatment period.

24. The method of claim 1 wherein the ultrasound energy is transmitted in one of either pulsed or continuous mode with varying intensity and frequency.

25. The method of claim 1 wherein the ultrasound energy delivered comprises of a frequency range between 20 KHz and 100 GHz.

26. The method of claim 1 wherein the said catheter also contains probes for central nervous system pressure and temperature monitoring.

27. A method of maintaining drainage catheter patency wherein ultrasound energy delivered through the catheter is used to dissolve the catheter lumen obstruction from hemorrhage and/or debris.

28. The method of claim 27 further comprising the steps of draining hemorrhage through a lumen in the said catheter with either a suction system or vacuum bulb or drainage bag attached to the distal end of the catheter.

29. The method of claim 27 wherein the said catheter shape is oval or rectangular.

30. The method of claim 27 wherein the said catheter is used as a drain following cranial or spinal surgery.

31. The method of claim 30 wherein the said cranial surgery is one of the following: craniotomy, burr hole, twist drill skull hole, or percutaneous skull hole.

32. The method of claim 30 wherein the said spinal surgery is one of the following: laminectomy, laminotomy, transforaminal or interlaminar percutaneous spinal placement

33. A method of treating hemorrhage in the intracranial or spinal subdural and/or subarachnoid space wherein ultrasound energy delivered through a catheter is used to dissolve and drain the said hemorrhage.

34. The method of claim 33 comprising the steps of: a) inserting the catheter into the hemorrhage through an opening in the skull or spine; b) delivering ultrasonic energy through the catheter; c) draining the hemorrhage through the catheter.

35. The method of claim 34 further comprising the step of delivering a blood clot lysis agent through the said catheter selected from one or more of the following: i) thrombolytics like streptokinase, urokinase, prourokinase, ancrod, tissue plasminogen activators (alteplase, anistreplase, tenecteplase, reteplase, duteplase), ii) hemolytic agents, iii) antiplatelet agents like GP IIb IIIa, aspirin, ticlopidine, clopidogrel, dipyridamole, iv) anticoagulants like heparin or warfarin, v) fibrinolytic agent like aspirin, vi) thrombolytic agent incorporated into micro-bubbles which can be ultrasonically activated after direct infusion into the blood clot.
